(19) United States
(12) Patent Application Publication
(10) Pub. No.: US 2009/0047260 A1
(43) Pub. Date: Feb. 19, 2009

(54) KERATIN BIOMATERIALS FOR CELL CULTURE AND METHODS OF USE

(75) Inventor: Mark E. Van Dyke, Winston-Salem, NC (US)

Correspondence Address: MYERS BIGEL SIBLEY & SAJOVEC, PO BOX 37428, RALEIGH, NC, 27627, US

(73) Assignee: Wake Forest University Health Sciences, Winston-Salem, NC (US)

(21) Appl. No.: 12/192,490

(22) Filed: Aug. 15, 2008

Related U.S. Application Data
(60) Provisional application No. 60/956,454, filed on Aug. 17, 2007.

(51) Int. Cl. A61F 2/00; C12N 5/02; A61K 35/12

(52) U.S. Cl. 424/93.7; 435/395; 435/403; 435/402; 424/423

(57) ABSTRACT

Provided herein are cell culture substrates and microcarriers that include a keratin, e.g., in porous particulate form. The substrate may be provided in or further includes a liquid carrier and/or viable cells. The keratin may be alpha kerateines, gamma kerateines, and combinations thereof, and may be in the form of a meta keratin. In some embodiments, the keratin is acidic or basic. Methods of administering cultured cells are also provided, including administering the cell culture substrates or microcarriers to a subject in need thereof. Kits are further provided, and may include a suitable container; a plurality of cell culture substrates or microcarriers as described herein packaged into said container; and optionally, instructions for use.

### RELATED APPLICATIONS

This application claims the benefit under 35 U.S.C. § 119(e) of U.S. Provisional Patent Application Ser. No. 60/956,454, filed Aug. 17, 2007, the disclosure of which is incorporated herein by reference in its entirety.

### FIELD OF THE INVENTION

The present invention is generally related to keratin-based biomaterials and the use thereof for culture and delivery of cells.

### BACKGROUND OF THE INVENTION

Cell culture of mammalian cells has long been used for the production of many vaccines and genetically engineered proteins. Attachment-dependent cells have historically been cultivated on the walls of roller bottles or non-agitated vessels such as tissue culture flasks, which are used in many laboratories. As the need has developed to provide large amounts of certain antiviral vaccines, genetically engineered proteins, and other cell-derived products, attempts have been made to develop new systems for large-scale production of cells. One solution has been to increase the growth surface area per unit vessel volume and to implement convenient and appropriate environmental controls. Some of these technologies involved the use of packed-glass beads, stacked plates, rotating multiple tubes, and roller bottles with spiral films inside.

Using microcarriers for cell culture increases the surface area of growth by allowing cells to grow as monolayers on the surface of small spheres or other globular micro-structures, or as multilayers in the pores of macroporous structures. First described in 1967 by van Wezel (van Wezel, A. L. “Growth of Cell-Strains and Primary Cells on Micro-carders in Homogeneous Culture” (1967) Nature 216:64-65), early microcarriers consisted of positively charged DEAE-dextran beads suspended in culture media in a stirred vessel. Cells would attach to the bead surface and grow as a monolayer.

Various other materials have been used for microcarriers and microcarrier and cell culture substrate coatings since van Wezel's DEAE-dextran beads (see, e.g., review in van der Velden-de Groot, Cytotechnology (1995) 18:51-56). However, new materials are needed in order to provide optimal cell culture conditions for various applications. Additionally, biocompatible microcarriers are needed that may be used directly in methods of treatment, without the need for cell harvesting.

### SUMMARY OF THE INVENTION

Provided herein are cell culture substrates that include a keratin, e.g., in porous particulate form. In some embodiments, the keratin is selected from the group consisting of: alpha kerateines, gamma kerateines, and combinations thereof. In some embodiments, the keratin comprises, consists of or consists essentially of a meta keratin. In some embodiments, the keratin is acidic or basic. In some embodiments, the substrate further includes a liquid carrier and/or viable cells.

Also provided are cell culture microcarriers that include a keratin in porous particulate form. In some embodiments, the microcarrier has an average diameter greater than 10 micrometers, and in some embodiments the microcarrier has an average diameter between 10 micrometers and 500 micrometers. In some embodiments, the keratin is selected from the group consisting of: alpha kerateines, gamma kerateines, and combinations thereof. In some embodiments, the keratin comprises, consists of or consists essentially of a meta keratin. In some embodiments, the keratin is acidic or basic. In some embodiments, the microcarrier further includes a liquid carrier and/or viable cells.

Methods of administering cultured cells are also provided, which include administering the cell culture substrates or microcarriers as described herein to a subject in need thereof. In some embodiments, the administering step is carried out by injection.

Kits are further provided, and in some embodiments include a suitable container; a plurality of cell culture substrates or microcarriers as described herein packaged into said container; and optionally, instructions for use. In some embodiments, cell culture substrates or microcarriers are packaged in said container in sterile form.

Another aspect of the present invention is the use of a cell culture substrate or microcarrier as described herein for the preparation of a composition or medicament for carrying out a method of treatment as described herein (e.g., cell replacement therapy), or for making an article of manufacture as described herein.

### BRIEF DESCRIPTION OF THE DRAWINGS

FIG. 1. Select binding domains found in known keratins. Peptide binding motifs are concentrated in the alpha keratins, particularly the acidic form.

FIG. 2. General schematic illustrating the production of various keratin derivatives from hair.

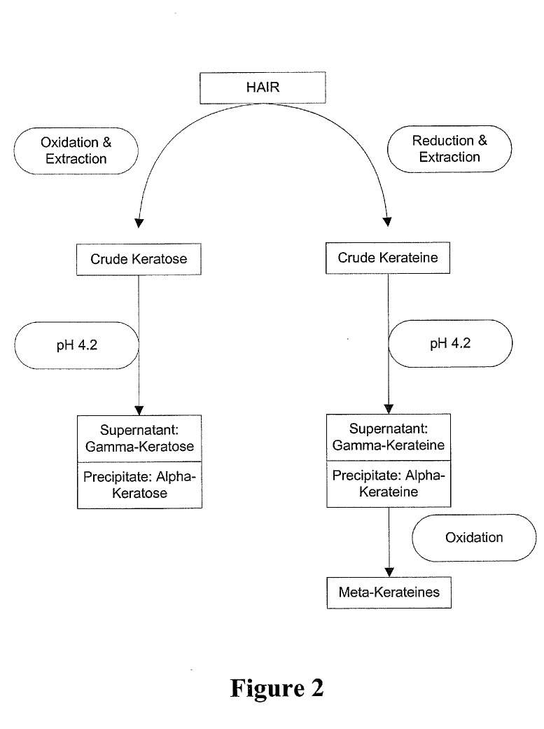

FIG. 3. Microscopy images at Day 7 of non-coated, collagen-coated and keratin-coated cell culture surfaces at 40×, 100× and 200×.

FIG. 4. Microscopy images at Day 14 of non-coated, collagen-coated and keratin-coated cell culture surfaces at 40×, 100× and 200×.

FIG. 5. Effect of cell density on cell growth (measured by cell number) in vitro at Day 7 of non-coated, collagen-coated and keratin-coated cell culture surfaces. Collagen and keratin were dissolved in PBS.

FIG. 6. Beta TC-6 cell growth at Day 7 in coatings prepared with different solutions.

FIG. 7. Percent adhesion of Beta TC-6 cells upon incubation for 2 and 6 hours in non-coated, collagen-coated and keratin-coated cell culture surfaces. Keratin was dissolved in distilled water, and collagen was dissolved in acetic acid.

FIGS. 8A-8B. Cell growth curves (wet-coating) over 7 days with non-coated, keratin-coated and collagen-coated cell culture surfaces. 8A: keratin and collagen were dissolved in PBS (initial cell density 20×10³/ml). 8B: keratin was dissolved in distilled water, and collagen was dissolved in acetic acid.

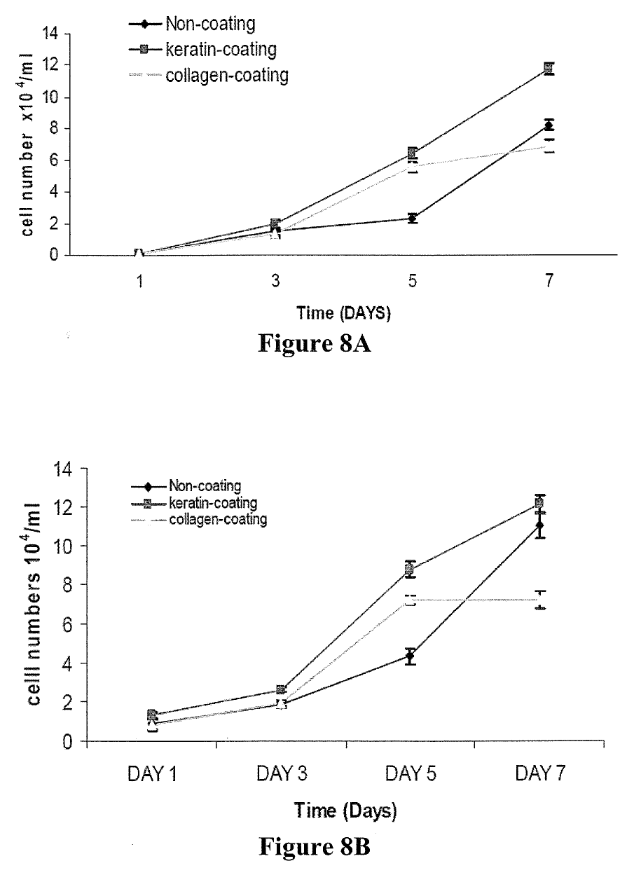

FIG. 9. Effect of keratin coating on insulin secretion of Beta TC-6 cells.

### DETAILED DESCRIPTION OF THE PREFERRED EMBODIMENTS

Described herein are keratin substrates useful in cell culture. In some embodiments the keratins are biocompatible, promote cell growth, promote cell adhesion and provide an excellent substrate for cell culture. The keratin substrates may also be used to deliver cells for, e.g., cell therapy applications.

The disclosures of all cited United States patent references are hereby incorporated by reference to the extent they are consistent with the disclosure herein. As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” refers to and encompasses any and all possible combinations of one or more of the associated listed items, as well as the lack of combinations when interpreted in the alternative (“or”).

### Definitions

“Cell culture” is the growth or proliferation of cells in vitro. “Cell” or “cells” as used herein may be any type of eukaryotic or prokaryotic cell, without limitation. Mammalian cells (including mouse, rat, dog, cat, monkey and human cells) are in some embodiments preferred, e.g., for tissue engineering applications. In some embodiments, cells are provided in or further include a liquid carrier. The liquid carrier can be in the form of a suspension, solution, or any other suitable form. Examples of suitable liquid carriers include, but are not limited to, water, aqueous solutions (e.g., phosphate buffer solution, citrate buffer solution, etc.), liquid media (e.g., modified Eagle's medium (“MEM”), Hanks' Balanced Salts, etc.), gels, and so forth, and in some embodiments may also include additional ingredients as desired.

Keratins are a family of proteins found in the hair, skin, and other tissues of vertebrates. Hair is a unique source of human keratins because it is one of the few human tissues that is readily available and inexpensive. Although other sources of keratins are acceptable feedstocks for the present invention, (e.g. wool, fur, horns, hooves, beaks, feathers, scales, and the like), human hair is preferred for use with human subjects because of its biocompatibility. The human hair can be end-cut, as one would typically find in a barber shop or salon.

“Keratin derivative” as used herein refers to any keratin fractionation, derivative, subfamily, etc., or mixtures thereof, alone or in combination with other keratin derivatives or other ingredients, including, but not limited to, alpha keratose, gamma keratose, alpha kerateine, gamma kerateine, meta keratin, keratin intermediate filaments, and combinations thereof, including the acidic and basic constituents thereof unless specified otherwise, along with variations thereof that will be apparent to persons skilled in the art in view of the present disclosure.

Proteins (such as growth factors) or other additives (such as antibiotics, anti-inflammatories, and modulators of the immune response) may also be added to the cell and/or keratin preparations at any time. Also, various treatments may be applied to enhance adherence of cells to the substrate and/or to each other. Appropriate treatments are described, for example, in U.S. Pat. No. 5,613,982. For example, collagen, elastin, fibronectin, laminin, or proteoglycans may be applied to the keratin substrates or microcarriers. The substrate or microcarrier can be impregnated with growth factors such as nerve growth factor (NGF), aFGF, bFGF, PDGF, TGFβ, VEGF, GDF-5/6/7, BMP-1/2/3/4/5/6/7/13/12/14, IGF-1, etc., or these agents may be provided in the liquid carrier (e.g., the culture medium).

Cells may be “attachment-dependent” (proliferating only after adhesion to a suitable culture surface or substrate), “attachment-independent” (able to proliferate without the need to attach to a surface or substrate), or both, and growth parameters may be adapted accordingly. For example, some animal cell types, such as lymphocytes, can grow in suspension, while others, including fibroblasts and epithelial and endothelial cells, are attachment-dependent and must attach to a surface and spread out in order to grow. Other cells can grow either in suspension or attached to a surface.

Cells that can be grown on the keratin substrates disclosed herein include, but are not limited to, eukaryotic cells and other microorganisms (e.g. yeast cells) such as stem and progenitor cells (whether embryonic, fetal, or adult), germ cells, somatic cells, etc., without limitation (See, e.g., U.S. Pat. No. 6,808,704 to Lanza et al.; U.S. Pat. No. 6,132,463 to Lee et al.; and U.S. Patent Application Publication No. 2005/0124003 to Atala et al.), as well as prokaryotic cells, including, but not limited to, bacteria (e.g., those that are genetically modified to produce specific biological molecules of interest such as therapeutic compounds).

“Cells of interest” are cells which are, or are intended to be, cultured using the methods disclosed herein. For example, cells of interest may be a particular type of cell isolated from a tissue or culture.

As used herein, “growth factors” include molecules that promote the regeneration, growth and survival of cells or tissue. Growth factors that are used in some embodiments of the present invention may be those naturally found in keratin extracts, or may be in the form of an additive, added to the keratin extracts or formed keratin substrates or microcarriers. Examples of growth factors include, but are not limited to, nerve growth factor (NGF) and other neurotrophins, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), epidermal growth factor (EGF), hepatocyte growth factor (HGF), granulocyte-colony stimulating factor (G-CSF), and granulocyte-macrophage colony stimulating factor (GM-CSF). There are many structurally and evolutionarily related proteins that make up large families of growth factors, and there are numerous growth factor families, e.g., the neurotrophins (NGF, BDNF, and NT3). The neurotrophins are a family of molecules that promote the growth and survival of, inter alia, nervous tissue. Examples of neurotrophins include, but are not limited to, nerve growth factor (NGF), brain-derived neurotrophic factor (BDNF), neurotrophin 3 (NT-3), and neurotrophin 4 (NT-4). See U.S. Pat. No. 5,843,914 to Johnson, Jr. et al.; U.S. Pat. No. 5,488,099 to Persson et al.; U.S. Pat. No. 5,438,121 to Barde et al.; U.S. Pat. No. 5,235,043 to Collins et al.; and U.S. Pat. No. 6,005,081 to Burton et al.

“Substrates” include porous, particulate, and non-porous (i.e., smooth) surfaces. Substrates may be a synthetic or natural material, and include living and non-living substrates. In some embodiments, a “substrate” includes, but is not limited to, a keratin composition (e.g., a microcarrier comprising, consisting of or consisting essentially of a keratin). In other embodiments, a substrate (e.g., glass, polystyrene) may be coated with a keratin composition. As appreciated by those of skill in the art, certain cell types may grown more readily on a substrate having a certain range of pore size and/or porosity, media and/or supplements, pH, etc.

“Subjects” are generally human subjects and include, but are not limited to, “patients.” The subjects may be male or female and may be of any race or ethnicity, including, but not limited to, Caucasian, African-American, African, Asian, Hispanic, Indian, etc. The subjects may be of any age, including newborn, neonate, infant, child, adolescent, adult, and geriatric.

Subjects may also include animal subjects, particularly mammalian subjects such as canines, felines, bovines, caprines, equines, ovines, porcines, rodents (e.g., rats and mice), lagomorphs, non-human primates, etc., for, e.g., veterinary medicine, laboratory research and/or pharmaceutical drug development purposes.

In some embodiments, methods of treatment are provided by administering a substrate (e.g., a keratin microcarrier) that further comprises cells. “Treat” refers to any type of treatment that imparts a benefit to a patient, e.g., a patient afflicted with or at risk for developing a disease (e.g., kidney disease). Treating includes actions taken and actions refrained from being taken for the purpose of improving the condition of the patient (e.g., the relief of one or more symptoms), delay in the onset or progression of the disease, etc.

Cells may be syngeneic (i.e., genetically identical or closely related, so as to minimize tissue transplant rejection), allogeneic (i.e., from a non-genetically identical member of the same species) or xenogeneic (i.e., from a member of a different species) with respect to a subject. Syngeneic cells include those that are autogeneic (i.e., from the patient to be treated) and isogeneic (i.e., a genetically identical but different subject, e.g., from an identical twin). Cells may be obtained from, e.g., a donor (either living or cadaveric) or derived from an established cell strain or cell line. For example, cells may be harvested from a donor (e.g., a potential recipient of a bioscaffold graft) using standard biopsy techniques known in the art.

“Microcarriers” are small, discrete particles employed to expand two-dimensional cell culture to three dimensions. See, e.g., U.S. Pat. No. 3,717,551 to Bizzini et al., U.S. Pat. No. 4,036,693 to Levine et al., U.S. Pat. No. 4,153,510 to Messing et al., U.S. Pat. No. 4,189,534 to Levine et al., U.S. Pat. No. 4,237,033 to Scattergood, U.S. Pat. No. 4,266,032 to Miller et al., U.S. Pat. No. 4,293,654 to Levine et al., U.S. Pat. No. 4,335,215 to Tolbert et al., U.S. Pat. No. 4,824,946 to Schwengers et al., U.S. Pat. No. 5,006,467 to Kusano et al., and U.S. Pat. No. 5,512,474 to Clapper et al. Microcarriers provide high surface area and can be utilized in stirred bioreactors, fluidized beds, packed columns, etc., to support high cell densities in liquid media.

Microcarriers can be ionic or non-ionic. Examples of ionic microcarriers include, but are not limited to, DEAE-Sephadex A50, low charge Sephadex, DEAE-cellulose, DEAE-cellulose fibers, polyacrylamide, polystyrene, derivatized polyacrylein microspheres in agarose, glass, glass-coated plastics, etc. Examples of non-ionic microcarriers include, but are not limited to, dextran beads with denatured collagen, gelatin (e.g., crosslinked with gluttaraldehyde, macroporous gelatin microcarriers, etc.), cellulose, polystyrene coated with collagen, polyethylene, polystyrol, polyurethane binder (e.g., with fibronectine factors), polyester fiber with collagen, etc. Ionic materials are generally used to manufacture smooth microcarriers, while non-ionic materials are generally used for macroporous carriers (see van der Velden-de Groot, Cytotechnology (1995) 18:51-56). For cells growing in suspension (e.g., attachment-independent), cells may be encapsulated in a microporous gel (e.g., agarose, gelatin, etc.).

In some embodiments, the keratin substrates or microcarriers have a pore size and/or porosity that is ideal for the infiltration and attachment of cells of interest (e.g., attachment-dependent cells). “Pore size” refers to the two-dimensional measurement of empty or void space present in the substrate, while porosity refers to the three-dimensional measurement of empty space or void volume per total volume. As a general guide, eukaryotic animal cells and plant cells are typically from 10 to 100 μm, and prokaryotic cells are typically from 0.1 to 10 μm in diameter. Upon enzymatic treatment (e.g., trypsinization), the cells typically to shrink to smaller spheres. As a general guide, after enzymatic treatment animal cells are typically from several micrometers to 30 micrometers.

In some embodiments, average pore sizes are large enough to accommodate an intact cell. For example, in some embodiments the resulting pore sizes are greater than 1 micron, and more preferably greater than 50 microns. In other embodiments, the pore size may be 100 microns or more. In some embodiments, the average pore size of keratin substrates or microcarriers developed using the processes described herein on bone tissue is from 400-1000 microns. In some embodiments, the ideal pore size of ligament, tendon, and meniscus tissues is from 100-1000 microns. In some embodiments, the average pore size is approximately 1.5 to 3 times the cell diameter of the cells of interest. In some embodiments, the average pore size is approximately three times a cell diameter of 1 to 30, 40, or 50 or more microns (i.e., 3 to 90, 120, or 150 or more microns).

In other embodiments, average pore sizes are not large enough to accommodate intact cells, and cells can attach only to the surface of the substrate. For example, the average pore size in some embodiments is less than 100, 70, 50, 20, 10, 1.0, or 0.5 microns.

In some embodiments, bulk porosity (void fraction) ranges from 50 to 99%. A preferred porosity is greater than 80%. A most preferred porosity is greater than 90%.

In some embodiments, keratin substrates or microcarriers are not water soluble. In other embodiments, they are “biostable,” meaning they are not broken down by typical cell secreted enzymes (e.g., matrix metalloproteases), making them suitable as substrates for long-term microcarrier cell cultures (e.g., from 3 or 6 months to a year or more).

### Preparation of Keratin Solutions and Substrates

Extracted keratin solutions are known to spontaneously self-assemble at the micron scale (Thomas H et al., Int J Biol Macromol 1986; 8:258-64; van de Löcht M, Melliand Textilberichte 1987; 10:780-6). This ability to self-assemble is particularly useful for cell culture substrates and microcarriers. Self-assembly results in a highly regular structure with reproducible architectures, dimensionality, and porosity. When the keratin is processed correctly, this ability to self-assemble can be preserved and used to create regular architectures on a size scale conducive to cellular infiltration and/or attachment. When keratins are hydrolyzed (e.g., with acids or bases), their molecular weight is reduced, and they lose the ability to self-assemble. Therefore, in some embodiments processing conditions that minimize hydrolysis are preferred.

Cellular recognition is also an important characteristic of biomaterials that seek to mimic the extracellular matrix (ECM). Such recognition is facilitated by the binding of cell surface integrins to specific amino acid motifs presented by the constituent ECM proteins. Predominant proteins include collagen and fibronectin, both of which have been extensively studied with regard to cell binding. Both proteins contain several regions that support attachment by a wide variety of cell types. It has been shown that in addition to the widely know Arginine-Glycine-Aspartic Acid (RGD) motif, the “X”-Aspartic Acid-“Y” motif on fibronectin is also recognized by the integrin α4β1, where X equals Glycine, Leucine, or Glutamic Acid, and Y equals Serine or Valine.

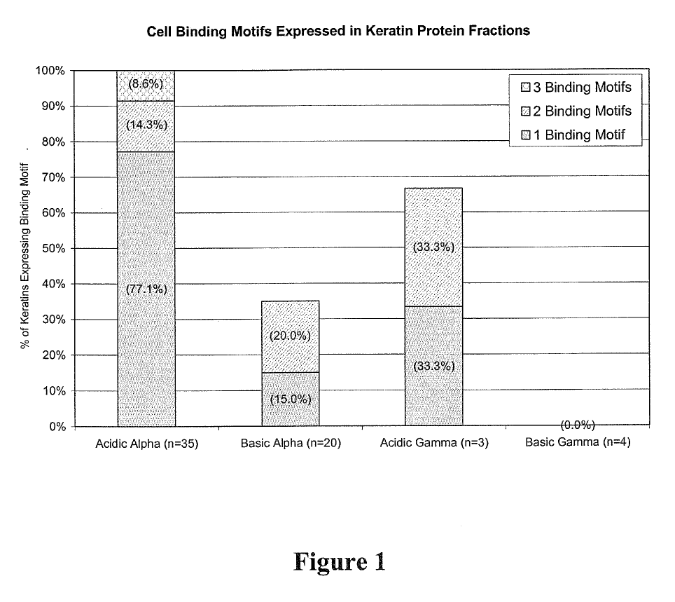

Keratin biomaterials derived from human hair also contain these binding motifs. A search of the NCBI protein database revealed sequences for 62 discrete, unique human hair keratin proteins. Of these, 55 are from the high molecular weight, low sulfur, alpha-helical family, and 7 are from the low molecular weight, high sulfur, globular family. The high molecular weight group of proteins is often referred to as the “alpha” keratins and is responsible for imparting toughness to human hair fibers. These alpha keratins have molecular weights greater than 50 kDa and an average cysteine (the main amino acid responsible for inter- and intramolecular protein bonding) content of 4.9 mole percent. The latter group of proteins is referred to as the “gamma” keratins is considered to aid in crosslinking the cortical proteins. These gamma keratins have a molecular weight of approximately 13 kDa and an average cysteine content of 8.7 mole %. Importantly, alpha and gamma proteins can be further sub-fractionated into acidic and basic fractions. Interestingly, peptide binding domains are concentrated in the alpha fraction, in particular the acidic alpha fraction. This group of proteins is relatively simple to isolate by precipitation and chromatographic methods in order to enhance cell attachment. FIG. 1 shows the general distribution of peptide binding motifs on the known human hair keratins. These binding sites are likely present on the surface of keratin biomaterials, as demonstrated by the finding of excellent cell adhesion onto processed keratin foams (see Tachibana A et al., J Biotech 2002; 93:165-70; Tachibana A et al., Biomaterials 2005; 26(3):297-302).

Other examples of natural polymers that may be utilized in a similar fashion to the disclosed keratin preparations include, but are not limited to, collagen, gelatin, fibronectin, vitronectin, laminin, fibrin, mucin, elastin, nidogen (entactin), proteoglycans, etc. (See,. e.g., U.S. Pat. No. 5,691,203 to Katsuen et al.).

Growth factors are known to be present in developing hair follicles (Jones C M et al., Development 1991; 111:531-42; Lyons K M et al., Development 1990; 109:833-44; Blessings M et al., Genes and Develop 1993; 7:204-15). In fact, more than 30 growth factors and cytokines are involved in the growth of a cycling hair follicle (Hardy M H, Trends Genet 1992; 8(2):55-61; Stenn K S et al., J Dermato Sci 1994; 7S:S109-24; Rogers G E, Int J Dev Biol 2004; 48(2-3):163-70). Many of these molecules have a pivotal role in the regeneration of a variety of tissues. It is highly probable that a number of growth factors become entrained within human hair when cytokines bind to stem cells residing in the bulge region of the hair follicle (Panteleyev A A et al., J Cell Sci 2001; 114:3419-31). Without wishing to be bound to any particular theory, it is thought that these growth factors are extracted along with the keratins from end-cut human hair. This is consistent with previous reports that many different types of growth factors are present in the extracts of various tissues, and that their activity is maintained even after chemical extraction. Observations such as these show mounting evidence that a number of growth factors may be present in end-cut human hair, and that the keratins may be acting as a highly effective delivery matrix of, inter alia, these growth factors.

Keratins can be extracted from human hair fibers by oxidation or reduction using known methods (See, e.g., Crewther W G et al. The chemistry of keratins, in Advances in protein chemistry 1965; 20:191-346). These methods typically employ a two-step process whereby the crosslinked structure of keratins is broken down by either oxidation or reduction. In these reactions, the disulfide bonds in cysteine amino acid residues are cleaved, rendering the keratins soluble. The cuticle is essentially unaffected by this treatment, so the majority of the keratins remain trapped within the cuticle's protective structure. In order to extract these keratins, a second step using a denaturing solution is employed. Alternatively, in the case of reduction reactions, these steps can be combined. Denaturing solutions known in the art include urea, transition metal hydroxides, surfactant solutions, and combinations thereof. Preferred methods use aqueous solutions of tris in concentrations between 0.1 and 1.0 M, and urea solutions between 0.1 and 10M, for oxidation and reduction reactions, respectively.

If one employs an oxidative treatment, the resulting keratins are referred to as “keratoses.” If a reductive treatment is used, the resulting keratins are referred to as “kerateines” (See Scheme 1).

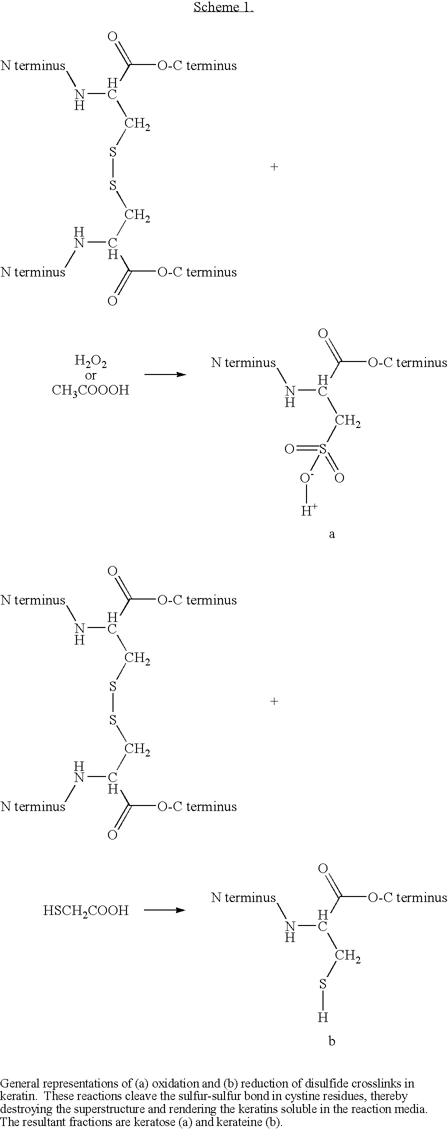

Crude extracts of keratins, regardless of redox state, can be further refined into “gamma” and “alpha” fractions, e.g., by isoelectric precipitation. High molecular weight keratins, or “alpha keratins,” (alpha helical), are thought to originate from the microfibrillar regions of the hair follicle, and typically range in molecular weight from about 40-85 kiloDaltons. Low molecular weight keratins, or “gamma keratins,” (globular), are thought to originate from the matrix regions of the hair follicle, and typically range in molecular weight from about 10-15 kiloDaltons. (See Crewther W G et al. The chemistry of keratins, in Advances in Protein Chemistry 1965; 20:191-346)

Even though alpha and gamma keratins possess unique properties, the properties of subfamilies of both alpha and gamma keratins can only be revealed through more sophisticated means of purification and separation. Additional properties that are beneficial to cell culture and cell delivery emerge and can be optimized upon further separation and purification of crude keratin extracts. Many protein purification techniques are known in the art, and range from the most simplistic, such as fractional precipitation, to the more complex, such as immunoaffinity chromatography. For extensive treatment of this subject, see Scopes R K (editor) Protein Purification: Principles and Practice (3rd ed. Sringer, New York 1993); Roe S, Protein Purification Techniques: A Practical Approach (2nd ed. Oxford University Press, New York 2001); Hatti-Kaul R and Mattiasson B, Isomation and Purification of Proteins (Marcel Dekker AG, New York 2003). For example, sub-families of acidic and basic keratin are separable by moving boundary electrophoresis. A preferred method of fractionation is ion exchange chromatography. We have discovered that these fractions possess unique properties, such as their differential effects on blood cell aggregation (See U.S. Patent Application Publication No. 2006/0051732).

In some embodiments,- the keratin derivative comprises, consists or consists essentially of a particular fraction or subfraction of keratin. The derivative in some embodiments may comprise, consist or consist essentially of at least 80, 90, 95 or 99 percent by weight of said fraction or subfraction (or more).

In some embodiments, the keratin derivative comprises, consists of, or consists essentially of acidic and/or basic, alpha and/or gamma keratose, where the keratose comprises, consists of or consists essentially of at least 80, 90, 95 or 99 percent by weight of acidic and/or basic, alpha and/or gamma keratose (or more).

In some embodiments, the keratin derivative comprises, consists of, or consists essentially of acidic and/or basic, alpha and/or gamma kerateine, where the kerateine comprises, consists of or consists essentially of at least 80, 90, 95 or 99 percent by weight of acidic and/or basic, alpha and/or gamma kerateine (or more).

For example, in some embodiments, the keratin derivative comprises, consists of or consists essentially of unfractionated alpha+gamma kerateines. In some embodiments, the keratin derivative comprises, consists of or consists essentially of acidic alpha+gamma kerateines. In some embodiments, the keratin derivative comprises, consists of or consists essentially of basic alpha+gamma kerateines.

In some embodiments, the keratin derivative comprises, consists of or consists essentially of unfractionated beta-keratose (e.g., derived from cuticle). In some embodiments, the keratin derivative comprises, consists of or consists essentially of basic beta-keratose. In some embodiments, the keratin derivative comprises, consists of or consists essentially of acidic beta-keratose.

Basic alpha kerateine is preferably produced by separating basic alpha kerateine from a mixture of acidic and basic alpha kerateine, e.g., by ion exchange chromatography, and optionally the basic alpha kerateine has an average molecular weight of from 10 to 100 or 200 kiloDaltons. More preferably, the average molecular weight is from 30 or 40 to 90 or 100 kiloDaltons. Optionally, but preferably, the process further includes the steps of re-dissolving said basic alpha-kerateine in a denaturing and/or buffering solution, optionally in the presence of a chelating agent to complex trace metals, and then re-precipitating the basic alpha kerateine from the denaturing solution. It will be appreciated by those of skill in the art that the composition preferably contains not more than 5, 2, 1, or 0.1 percent by weight of acidic alpha kerateine, or less.

The acidic alpha kerateine is preferably produced by a reciprocal of the foregoing technique: that is, by separating and retaining acidic alpha kerateine from a mixture of acidic and basic alpha kerateine, e.g., by ion exchange chromatography, and optionally the acidic alpha kerateine has an average molecular weight of from 5 or 10 to 100 or 200 kiloDaltons. Optionally, but preferably, the process further comprises the steps of re-dissolving said acidic alpha-kerateine in a denaturing and/or buffering solution), optionally in the presence of a chelating agent to complex trace metals, and then re-precipitating the basic alpha kerateine from the denaturing solution. It will be appreciated that the composition preferably contains not more than 5, 2, 1, or 0.1 percent by weight of basic alpha kerateine, or less.

Basic and acidic fractions of other kerateines (e.g., gamma kerateine) can be prepared in like manner as described above for basic and acidic alpha kerateine. Gamma keratins are typically precipitated in a non-solvent such as ethanol.

Keratin materials are derived from any suitable source, including, but not limited to, wool and human hair. In one embodiment keratin is derived from end-cut human hair, obtained from barbershops and salons. The material is washed in hot water and mild detergent, dried, and extracted with a nonpolar organic solvent (typically hexane or ether) to remove residual oil prior to use.

Preparation of Kerateines. Kerateine fractions can be obtained using a combination of the methods of Bradbury and Chapman (J. Bradbury et al., Aust. J. Biol. Sci. 17, 960-72 (1964)) and Goddard and Michaelis (D. Goddard et al., J. Biol. Chem. 106, 605-14 (1934)). Essentially, the cuticle of the hair fibers is removed ultrasonically in order to avoid excessive hydrolysis and allow efficient reduction of cortical disulfide bonds in a second step. The hair is placed in a solution of dichloroacetic acid and subjected to treatment with an ultrasonic probe. Further refinements of this method indicate that conditions using 80% dichloroacetic acid, solid to liquid of 1:16, and an ultrasonic power of 180 Watts are optimal (H. Ando et al., (1975) Sen'i Gakkaishi 31(3), T81-85). Solid fragments are removed from solution by filtration, rinsed and air dried, followed by sieving to isolate the hair fibers from removed cuticle cells.

In some embodiments, following ultrasonic removal of the cuticle, alpha and gamma kerateines are obtained by reaction of the denuded fibers with mercaptoethanol. Specifically, a low hydrolysis method is used at acidic pH (E. Thompson et al., Aust. J. Biol. Sci. 15, 757-68 (1962)). In a typical reaction, hair is extracted for 24 hours with 4M mercaptoethanol that has been adjusted to pH 5 by addition of a small amount of potassium hydroxide in deoxygenated water containing 0.02M acetate buffer and 0.001M surfactant.

The solution is filtered and the alpha kerateine fraction precipitated by addition of mineral acid to a pH of approximately 4. The alpha kerateine is separated by filtration, washed with additional acid, followed by dehydration with alcohol, and then dried under vacuum. Increased purity is achieved by re-dissolving the kerateine in a denaturing solution such as urea solutions between 0.1 and 10M (e.g., 7M urea), aqueous ammonium hydroxide solution, or 20 mM tris buffer solution, re-precipitating, re-dissolving, dialyzing against deionized water, and re-precipitating at pH 4.

The gamma kerateine fraction remains in solution at pH 4 and is isolated by addition to a water-miscible organic solvent such as alcohol, followed by filtration, dehydrated with additional alcohol, and dried under vacuum. Increased purity can be achieved by re-dissolving the kerateine in a denaturing solution such as 7M urea, aqueous ammonium hydroxide solution, or 20 mM tris buffer solution, reducing the pH to 4 by addition of a mineral acid, removing any solids that form, neutralizing the supernatant, re-precipitating the protein with alcohol, re-dissolving, dialyzing against deionized water, and reprecipitating by addition to alcohol. The amount of alcohol consumed in these steps can be minimized by first concentrating the keratin solution by distillation.

In an alternate method, the kerateine fractions are obtained by reacting the hair with an aqueous solution of sodium thioglycolate. A preferred method for the production of kerateines is by reduction of the hair with thioglycolic acid or beta-mercaptoethanol. A most preferred reductant is thioglycolic acid (TGA). Preferred concentrations range from 1 to 10M, the most preferred being approximately 1.0M. Those skilled in the art will recognize that slight modifications to the concentration can be made to effect varying degrees of reduction, with concomitant alterations in pH, reaction time, temperature, and liquid to solid ratio. A preferred pH is from 8 to 11.5, or from 8 to 11, or from 9 to 11. A most preferred pH is 10, or 10.2. The pH of the reduction solution is altered by addition of base. Preferred bases include transition metal hydroxides, sodium hydroxide, and ammonium hydroxide. A most preferred base is sodium hydroxide. The pH adjustment is effected by dropwise addition of a saturated solution of sodium hydroxide in water to the reductant solution. A preferred reduction temperature is between 0 and 100° C. A most preferred reduction temperature is 37° C. A preferred reduction time is between 0.5 and 24 hours. A most preferred reduction time is 12 hours. A preferred liquid to solid ratio is from 5 to 100:1. A most preferred ratio is 20:1. Unlike the previously described oxidation reaction, reduction is carried out at basic pH. That being the case, keratins are highly soluble in the reduction media and are expected to be extracted. The reduction solution is therefore combined with the subsequent extraction solutions and processed accordingly.

Reduced keratins are not as hydrophilic as their oxidized counterparts. As such, reduced hair fibers will not swell and split open as will oxidized hair, resulting in relatively lower yields. Another factor affecting the kinetics of the reduction/extraction process is the relative solubility of kerateines. The relative solubility rankings in water is gamma-keratose>alpha-keratose>gamma-kerateine>alpha-kerateine from most to least soluble. Consequently, extraction yields from reduced hair fibers are not as high. This being the case, subsequent extractions are conducted with additional reductant plus denaturing agent solutions. Preferred solutions for subsequent extractions include TGA plus urea, TGA plus tris base, or TGA plus sodium hydroxide. After extraction, crude fractions of alpha- and gamma-kerateine can be isolated using the procedures described for keratoses. However, precipitates of gamma- and alpha-kerateine re-form their cystine crosslinks upon exposure to oxygen. Precipitates must therefore be re-dissolved quickly to avoid insolubility during the purification stages, or precipitated in the absence of oxygen.

Residual reductant and denaturing agents can be removed from solution by dialysis. Typical dialysis conditions are 1 to 2% solution of kerateines dialyzed against DI water for 24 to 72 hours. Those skilled in the art will recognize that other methods exist for the removal of low molecular weight contaminants in addition to dialysis (e.g. microfiltration, chromatography, and the like). The use of tris base is only required for initial solubilization of the kerateines. Once dissolved, the kerateines are stable in solution without the denaturing agent. Therefore, the denaturing agent can be removed without the resultant precipitation of kerateines, so long as the pH remains at or above neutrality. The final concentration of kerateines in these purified solutions can be adjusted by the addition/removal of water.

Regardless of the form of the keratin (i.e. keratoses or kerateines), several different approaches to further purification can be employed to keratin solutions. Care must be taken, however, to choose techniques that lend themselves to keratin's unique solubility characteristics. One of the most simple separation technologies is isoelectric precipitation. In this method, proteins of differing isoelectric point can be isolated by adjusting the pH of the solution and removing the precipitated material. In the case of keratins, both gamma- and alpha-forms are soluble at pH>6.0. As the pH falls below 6, however, alpha-keratins begin to precipitate. Keratin fractions can be isolated by stopping the precipitation at a given pH and separating the precipitate by centrifugation and/or filtration. At a pH of approximately 4.2, essentially all of the alpha-keratin will have been precipitated. These separate fractions can be re-dissolved in water at neutral pH, dialyzed, concentrated, and reduced to powders by lyophilization or spray drying. However, kerateine fractions must be stored in the absence of oxygen or in dilute solution to avoid crosslinking.

Another general method for separating keratins is by chromatography. Several types of chromatography can be employed to fractionate keratin solutions including size exclusion or gel filtration chromatography, affinity chromatography, isoelectric focusing, gel electrophoresis, ion exchange chromatography, and immunoaffinity chromatography. These techniques are well known in the art and are capable of separating compounds, including proteins, by the characteristics of molecular weight, chemical functionality, isoelectric point, charge, or interactions with specific antibodies, and can be used alone or in any combination to effect high degrees of separation and resulting purity.

A preferred purification method is ion exchange (IEx) chromatography. IEx chromatography is particularly suited to protein separation owning to the amphiphilic nature of proteins in general and keratins in particular. Depending on the starting pH of the solution, and the desired fraction slated for retention, either cationic or anionic IEx (CIEx or AIEx, respectively) techniques can be used. For example, at a pH of 6 and above, both gamma- and alpha-keratins are soluble and above their isoelectric points. As such, they are anionic and can be bound to an anionic exchange resin. However, it has been discovered that a sub-fraction of keratins does not bind to a weakly anionic exchange resin and instead passes through a column packed with such resin. A preferred solution for AIEx chromatography is purified or fractionated keratin, isolated as described previously, in purified water at a concentration between 0 and 5 weight/volume %. A preferred concentration is between 0 and 4 w/v %. A most preferred concentration is approximately 2 w/v %. It is preferred to keep the ionic strength of said solution initially quite low to facilitate binding to the AIEx column. This is achieved by using a minimal amount of acid to titrate a purified water solution of the keratin to between pH 6 and 7. A most preferred pH is 6 for keratoses and 7 for kerateines. This solution can be loaded onto an AIEx column such as DEAE-Sepharose® resin or Q-Sepharose® resin columns. A preferred column resin is DEAE-Sepharose® resin. The solution that passes through the column can be collected and further processed as described previously to isolate a fraction of acidic keratin powder.

In some embodiments the activity of the keratin matrix is enhanced by using an AIEx column to produce the keratin that may be useful for, inter alia, promoting cell adhesion. Without wishing to be bound to any particular theory, it is thought that charged substrates promotes cell attachment. Though many cells have a negative surface charge, they attach to surfaces that are negatively as well as positively charged (see, e.g., van der Velden-de Groot “Microcarrier technology, present status and perspective” (1995) Cytotechnology 18:51-56).

Another fraction binds readily, and can be washed off the column using salting techniques known in the art. A preferred elution medium is sodium chloride solution. A preferred concentration of sodium chloride is between 0.1 and 2M. A most preferred concentration is 2M. The pH of the solution is preferred to be between 6 and 12. A most preferred pH is 12. In order to maintain stable pH during the elution process, a buffer salt can be added. A preferred buffer salt is Trizma® base. Those skilled in the art will recognize that slight modifications to the salt concentration and pH can be made to effect the elution of keratin fractions with differing properties. It is also possible to use different salt concentrations and pH's in sequence, or employ the use of salt and/or pH gradients to produce different fractions. Regardless of the approach taken, however, the column eluent can be collected and further processed as described previously to isolate fractions of basic keratin powders.

A complimentary procedure is also feasible using CIEx techniques. Namely, the keratin solution can be added to a cation exchange resin such as SP Sepharose® resin (strongly cationic) or CM Sepharose® resin (weakly cationic), and the basic fraction collected with the pass through. The retained acid keratin fraction can be isolated by salting as previously described.

Meta kerateines. Kerateines have labile sulfur residues. During the creation of the kerateines, cystine is converted to cysteine, which can be a source of further chemical modifications. One such useful reaction is oxidative sulfur-sulfur coupling. This reaction simply converts the cysteine back to cystine and reforms the crosslinks between proteins. Crosslinking gamma or alpha kerateine fractions, or a combination of both, produces meta-kerateines. This is a useful reaction to increase the molecular weight of kerateines, which in turn will modify their bulk properties. Increasing molecular weight influences material properties such a viscosity, dry film strength, gel strength, etc. Additionally, water solubility can be modified through the production of meta kerateines. The high crosslink density of meta kerateines renders these biomaterials essentially insoluble in aqueous media, making them amenable to applications where preservation of material integrity in such media is preferred.

Meta keratins can be derived from the gamma or alpha fractions, or a combination of both. Oxidative re-crosslinking of the kerateines is affected by addition of an oxidizing agent such as peracetic acid or hydrogen peroxide to initiate oxidative coupling reactions of cysteine groups. A preferred oxidizing agent is oxygen. This reaction can be accomplished simply by bubbling oxygen through the kerateine solution or by otherwise exposing the sample to air. Optimizing the molecular weight through the use of meta-keratins allows formulations to be optimized for a variety of properties including viscosity, film strength and elasticity, fiber strength, and hydrolytic susceptibility. Crosslinking in air works to improve biocompatibility by providing biomaterial with a minimum of foreign ingredients.

Basically, in some embodiments the kerateine is dissolved in a denaturing solution such as 7M urea, aqueous ammonium hydroxide solution, or 20 mM tris buffer solution. The progress of the reaction is monitored by an increase in molecular weight as measured using SDS-PAGE. Oxygen is continually bubbled through the reaction solution until a doubling or tripling of molecular weight is achieved. The pH of the denaturing solution can be adjusted to neutrality to avoid hydrolysis of the proteins by addition of mineral acid.

Optimizing the molecular weight through the use of meta-keratins allows formulations to be optimized for a variety of properties including viscosity, film strength and elasticity, fiber strength, and hydrolytic susceptibility. In some embodiments, crosslinking in air may improve biocompatibility by providing biomaterials with a minimum of foreign ingredients.

Keratin intermediate filaments. IFs of human hair fibers are obtained using the method of Thomas and coworkers (H. Thomas et al., Int. J. Biol. Macromol. 8, 258-64 (1986)). This is essentially a chemical etching method that reacts away the keratin matrix that serves to “glue” the IFs in place, thereby leaving the IFs behind. In a typical extraction process, swelling of the cuticle and sulfitolysis of matrix proteins is achieved using 0.2M Na₂SO₃, 0.1M Na₂O₆S₄ in 8M urea and 0.1M Tris-HCl buffer at pH 9. The extraction proceeds at room temperature for 24 hours. After concentrating, the dissolved matrix keratins and IFs are precipitated by addition of zinc acetate solution to a pH of approximately 6. The IFs are then separated from the matrix keratins by dialysis against 0.05M tetraborate solution. Increased purity is obtained by precipitating the dialyzed solution with zinc acetate, redissolving the IFs in sodium citrate, dialyzing against distilled water, and then freeze drying the sample.

Further discussion of keratin preparations are found in U.S. Patent Application Publication 2006/0051732 (Van Dyke), which is incorporated by reference herein.

Formulations. Dry powders may be formed of keratin preparations as described above in accordance with known techniques such as freeze drying (lyophilization). In some embodiments, compositions of the invention may be produced by mixing such a dry powder composition form with an aqueous solution to produce a composition having an electrolyte solution with a keratin solubilized therein. The mixing step can be carried out at any suitable temperature, typically room temperature, and can be carried out by any suitable technique such as stirring, shaking, agitation, etc. The salts and other constituent ingredients of the electrolyte solution (e.g., all ingredients except the keratin derivative and the water) may be contained entirely in the dry powder, entirely within the aqueous composition, or may be distributed between the dry powder and the aqueous composition. For example, in some embodiments, at least a portion of the constituents of the electrolyte solution is contained in the dry powder.

The formation of a substrate or microcarrier including keratin materials such as described above can be carried out in accordance with techniques long established in the field or variations thereof that will be apparent to those skilled in the art. In some embodiments, the keratin preparation is dried and rehydrated prior to use. See, e.g., U.S. Pat. No. 2,413,983 to Lustig et al., U.S. Pat. No. 2,236,921 to Schollkipf et al., and U.S. Pat. No. 3,464,825 to Anker. In some embodiments, lyophilized material is rehydrated with a suitable solvent, such as water or phosphate buffered saline (PBS). The material can be sterilized, e.g., by γ-irradiation (800 krad) using a ⁶⁰Co source. Other suitable methods of forming keratin matrices include, but are not limited to, those found in U.S. Pat. No. 6,270,793 (Van Dyke et al.), U.S. Pat. No. 6,274,155 (Van Dyke et al.), U.S. Pat. No. 6,316,598 (Van Dyke et al.), U.S. Pat. No. 6,461,628 (Blanchard et al.), U.S. Pat. No. 6,544,548 (Siller-Jackson et al.), and U.S. Pat. No. 7,01,987 (Van Dyke).

In some composition embodiments, the keratin preparations (particularly alpha and/or gamma kerateine and alpha and/or gamma keratose) have an average molecular weight of from about 10 to 70 or 85 or 100 kiloDaltons. Other keratin derivatives, particularly meta-keratins, may have higher average molecular weights, e.g., up to 200 or 300 kiloDaltons.

The keratin derivative composition or formulation may optionally contain one or more active ingredients such as one or more growth factors (e.g., in an amount ranging from 0.000000001, 0.000000005, or 0.00000001, to 0.00000001, 0.00000005, or 0.0000001 percent by weight of the composition that comprises the keratin) to facilitate cell or tissue adhesion and/or proliferation, etc. Examples of suitable active ingredients include, but are not limited to, nerve growth factor, vascular endothelial growth factor, fibronectin, fibrin, laminin, acidic and basic fibroblast growth factors, testosterone, ganglioside GM-1, catalase, insulin-like growth factor-I (IGF-I), platelet-derived growth factor (PDGF), neuronal growth factor galectin-1, and combinations thereof. See, e.g., U.S. Pat. No. 6,506,727 to Hansson et al. and U.S. Pat. No. 6,890,531 to Horie et al.

For example, nerve growth factor (NGF) can be added to the keratin composition in an amount effective to promote the regeneration, growth and survival of various tissues. The NGF is provided in concentrations ranging from 0.1 ng/mL to 1000 ng/mL. More preferably, NGF is provided in concentrations ranging from 1 ng/mL to 100 ng/mL, and most preferably 10 ng/mL to 100 ng/mL. See U.S. Pat. No. 6,063,757 to Urso.

The compositions, substrates and/or microcarriers are preferably sterile. In some embodiments, microcarriers are sterile filtered and processed aseptically, or terminally sterilized using ethylene oxide, e-beam, gamma, or other low temperature method (i.e. <50° C.).

The composition may be provided preformed and aseptically packaged in a suitable container, such as a flexible polymeric bag or bottle, or a foil container, or may be provided as a kit of sterile dry powder in one container and sterile aqueous solution in a separate container for mixing just prior to use. When provided pre-formed and packaged in a sterile container the composition preferably has a shelf life of at least 4 or 6 months (up to 2 or 3 years or more) at room temperature, prior to substantial loss of viscosity (e.g., more than 10 or 20 percent) and/or structural integrity of the keratin substrate or microcarrier.

Applications for the cell culture substrates and microcarriers include, but are not limited to, culturing bacteria, yeast, insect cells and animal (e.g., human) cells, e.g., for production of cells for therapy, vaccines and vectors, natural and recombinant proteins, antibodies, expansion and differentiation of stem cells, etc.

Microcarrier preparations. Kerateine (e.g., alpha, gamma or meta) solutions can be formed into microcarriers using a variety of techniques. Particles of kerateine can be produces in a variety of sizes and shapes, with varying degrees of porosity, hardness, surface chemistry, size and shape by changing the relative amounts of alpha and gamma fractions. Microparticle production techniques include spray drying, emulsion polymerization, and lyophilization followed by grinding. Specific sizes of microcarriers can be obtained by a number of sorting techniques known in the art such as sieving.

In addition to microcarriers formed from kerateine, microcarriers may be formed from ionic or non-ionic microcarriers (e.g., as listed above) and coated with kerateine. Alternatively, microcarriers may be formed from kerateine and coated with, e.g., collagen, gelatin, amino acids, etc.

In some embodiments, microcarriers have an average diameter greater than 10 μm are preferred, and those between 10 μm and 500 μm are most preferred (measured by, e.g., scanning electronic microscopy, light scattering techniques, etc.). In some embodiments, microcarriers have a relative density such that they can be maintained in suspension in a desired liquid (e.g., water, media, etc.) with gentle stirring, e.g., suspendable without shear that would harm or alter the cells.

Smaller sizes can be obtained using spray drying (e.g., 10-50 μm), while larger sizes can be produced using emulsion polymerization or grinding/sorting. Smaller particles are more easily suspended in media because they have a slower sedimentation rate, making them better suited for stirred bioreactor applications. Larger particles have higher sedimentation rates and are better suited for fluidized bed and packed column applications. In some embodiments, crosslinked kerateines are not water soluble, nor can they be broken down by typical cell secreted enzymes (e.g., matrix metalloproteases), making them suitable as substrates for long-term microcarrier cell cultures.

In some embodiments, physical properties of the keratin microcarriers are controlled by composition (e.g., alpha:gamma keratin ratio) and/or processing (e.g., spray drying, emulsion) techniques. Physical properties include, but are not limited to, pore size, porosity, hardness, size and shape of the microcarriers.

In some embodiments, biological properties (e.g., cell attachment) are controlled by composition and/or processing techniques. For example, microcarriers made with kerateine particles provide a surrogate extracellular matrix environment. Keratins possess many peptide binding motifs that are specific to the integrin receptors found on many cell types. Unlike conventional microcarriers, in some embodiments kerateines contain numerous regulatory molecules that are essential for cell function. As such they are useful to grow cells in high density. Applications include, but are not limited to, production of cells for therapy, vaccines and vectors, natural and recombinant proteins, antibodies, and expansion and differentiation of stem cells.

In further embodiments, the microcarriers may be weighted to achieve the desired specific gravity (see U.S. Pat. No. 4,861,714 to Dean, Jr. et al.). In other embodiments, keratin substrates or microcarriers are modified to produce the desired charge capacity (see U.S. Pat. Nos. 4,293,654 and 4,189,534 to Levine et al.).

Keratin coatings. In addition, any suitable substrate may be coated or treated with keratin materials or keratin derivatives as described herein to promote the adhesion of cells for cell culture.

The substrate may be formed from any suitable material, including but not limited to organic polymers (including stable polymers and biodegradable or bioerodable polymers), natural materials (e.g., collagen), metals (e.g., platinum, gold, stainless steel, etc.) inorganic materials such as silicon, glass, etc., and composites thereof. For example, styrene beads may be coated with keratin preparations.

Coating of the substrate may be carried out by any suitable means, such as spray coating, dip coating, or the like. In some embodiments, steps may be taken to couple or covalently couple the keratin to the substrate such as with a silane coupling agent, if so desired. The keratin derivative may be subsequently coated with another material, and/or other materials may be co-deposited with the keratin derivative, such as one or more additional active agents, stabilizers, coatings, etc.

The chemistry of keratins can be utilized to optimize the properties of keratin-based coatings. Alpha and gamma keratoses have inert sulfur residues. The oxidation reaction is a terminal step and results in the conversion of cystine residues into two non-reactive sulfonic acid residues. Kerateines, on the other hand, have labile sulfur residues. During the creation of the kerateines, cystine is converted to cysteine, which can be a source of further chemical modifications (See Scheme 1 above). One such useful reaction is oxidative sulfur-sulfur coupling. This reaction simply converts the cysteine back to cystine and reforms the crosslinks between proteins. This is a useful reaction for increasing the molecular weight of the gamma or alpha fraction of interest, which in turn will modify the bulk properties of the material. Increasing molecular weight influences material properties such as viscosity, dry film strength, gel strength, etc. Such reformed kerateines are referred to as meta keratins.

Methods of treatment. Because keratins are biocompatible, in some embodiments colonized microcarriers can be used directly for therapy such as an injectable (e.g., for cardiac regeneration) or as a surface treatment (e.g., for skin wounds). Keratin substrates or microcarriers may be administered to a subject in need thereof, with or without prior seeding or attachment of cultured cells. Formulations of the invention include those for parenteral administration (e.g., subcutaneous, intramuscular, intradermal, intravenous, intra-arterial, intraperitoneal injection) or implantation. In one embodiment, administration is carried out intravascularly, either by simple injection, or by injection through a catheter positioned in a suitable blood vessel, such as a renal artery. In some embodiments, administration of keratin substrates or microcarriers is carried out by “infusion,” whereby compositions are introduced into the body through a vein (e.g., the portal vein). In another embodiment, administration is carried out as a graft to an organ or tissue to be augmented as discussed above, e.g., kidney and/or liver.

Substrates or microcarriers may also be delivered systemically. In further embodiments, cells are delivered to certain tissues (e.g., the liver), but the outcome of the functional effects of the delivery will be systemic (e.g., microcarriers seeded with cells producing hormones). See, e.g., the “Edmonton protocol,” an established delivery method, where cells are infused into a patient's portal vein (Shapiro et al. (2000) N Engl J Med 343:230-238).

According to some embodiments, the cells administered to the subject may be syngeneic (i.e., genetically identical or closely related, so as to minimize tissue transplant rejection), allogeneic (i.e., from a non-genetically identical member of the same species) or xenogeneic (i.e., from a member of a different species), as above, with respect to the subject being treated, depending upon other steps such as the presence or absence of encapsulation or the administration of immune suppression therapy of the cells. Syngeneic cells include those that are autogeneic (i.e., from the subject to be treated) and isogeneic (i.e., a genetically identical but different subject, e.g., from an identical twin). Cells may be obtained from, e.g., a donor (either living or cadaveric) or derived from an established cell strain or cell line. As an example of a method that can be used to obtain cells from a donor (e.g., a potential recipient of a bioscaffold graft), standard biopsy techniques known in the art may be employed. Alternatively, cells may be harvested from the subject, expanded/selected in vitro, and reintroduced into the same subject (i.e., autogeneic).

In some embodiments, cells are administered in a therapeutically effective amount. The therapeutically effective dosage of cells will vary somewhat from subject to subject, and will depend upon factors such as the age, weight, and condition of the subject and the route of delivery. Such dosages can be determined in accordance with procedures known to those skilled in the art. In general, in some embodiments, a dosage of 1×10⁵, 1×10⁶ or 5×10⁶ up to 1×10⁷, 1×10⁸ or 1×10⁹ cells or more per subject may be given, administered together at a single time or given as several subdivided administrations. In other embodiments a dosage of between 1-100×10⁸ cells per kilogram subject body weight can be given, administered together at a single time or given as several subdivided administration. Of course, follow-up administrations may be given if necessary.

In further embodiments, if desired or necessary, the subject may be administered an agent for inhibiting transplant rejection of the administered cells, such as rapamycin, azathioprine, corticosteroids, cyclosporin and/or FK506, in accordance with known techniques. See, e.g., R. Calne, U.S. Pat. Nos. 5,461,058, 5,403,833 and 5,100,899; see also U.S. Pat. Nos. 6,455,518, 6,346,243 and 5,321,043. Some embodiments use a combination of implantation and immunosuppression, which minimizes rejection.

Kits are also provided, where the microcarriers described herein are provided in a suitable container (e.g. a plastic or glass bottle, sterile ampule, etc.), optionally packaged in sterile form. The microcarriers may be provided as a powder, or in an aqueous liquid, and may be provided in different volumes for specific cell densities. For example, microcarriers in some embodiments are packaged in alcohol (e.g., ethanol, propanol, etc.) for long-term sterility.

The present invention is explained in greater detail in the following non-limiting Examples.

### EXAMPLE 1

### Crude Kerateine Samples

Kerateine fractions were obtained using a modification of the method described by Goddard and Michaelis. Briefly, the hair was reacted with an aqueous solution of 1M TGA at 37° C. for 24 hours. The pH of the TGA solution had been adjusted to pH 10.2 by dropwise addition of saturated NaOH solution. The extract solution was filtered to remove the reduced hair fibers and retained. Additional keratin was extracted from the fibers by sequential extractions with 1000 mL of 10 mM TGA at pH 10.2 for 24 hours, 1000 mL of 10 mM TGA at pH 10.2 for 24 hours, and DI water at pH 10.2 for 24 hours. After each extraction, the solution was centrifuged, filtered, and added to the dialysis system. Eventually, all the extracts were combined and dialyzed against DI water with a 1 KDa nominal low molecular weight cutoff membrane. The solution was concentrated, titrated to pH 7, and stored at approximately 5% total protein concentration at 4° C. Alternately, the concentrated solution could be lyophilized and stored frozen and under nitrogen.

### EXAMPLE 2

### Ion Exchange Chromatography

Kerateines have a propensity to crosslink in air, so oxygen free processing is used. Just prior to fractionation, kerateine samples are titrated to pH 6 by careful addition of dilute HCl solution. The samples are loaded onto a 200 mL flash chromatography column containing either DEAE-Sepharose® (weakly anionic) or Q-Sepharose® (strongly anionic) exchange resin (50-100 mesh; Sigma-Aldrich, Milwaukee, Wis.) with gentle pressure and the flow through collected (acidic keratin). A small volume of 10 mM Trizma® base (approximately 200 mL) at pH 6 is used to completely wash through the sample. Basic kerateine is eluted from the column with 100 mM tris base plus 2M NaCl at pH 12. Each sample is separately neutralized and dialyzed against DI water using tangential flow dialysis with a LMWCO of 1 KDa, concentrated by rotary evaporation, and freeze dried.

### EXAMPLE 3

### Cell Culture With Keratin-coated Tissue Culture Dishes

To test the feasibility of using keratin protein as coating material for functional cell growth, polystyrene tissue culture dishes were coated using a keratin solution of unfractionated alpha+gamma kerateine (100 μg/ml), and compared to dished coated with collagen (100 μg/ml) and a non-coated dish.

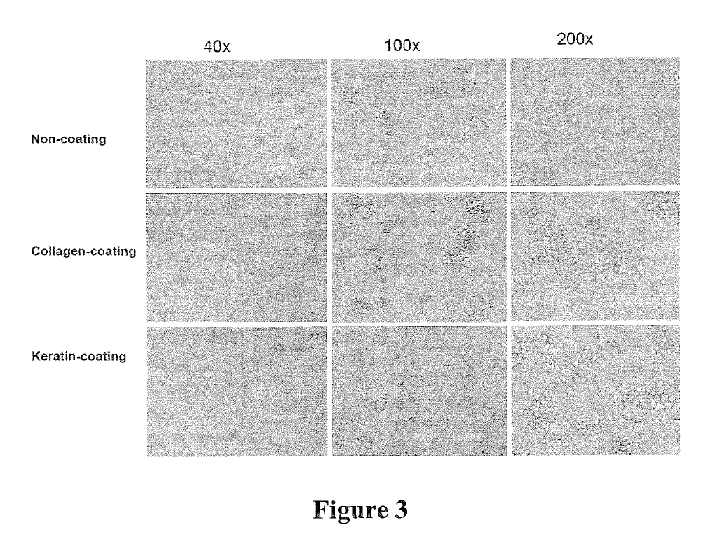

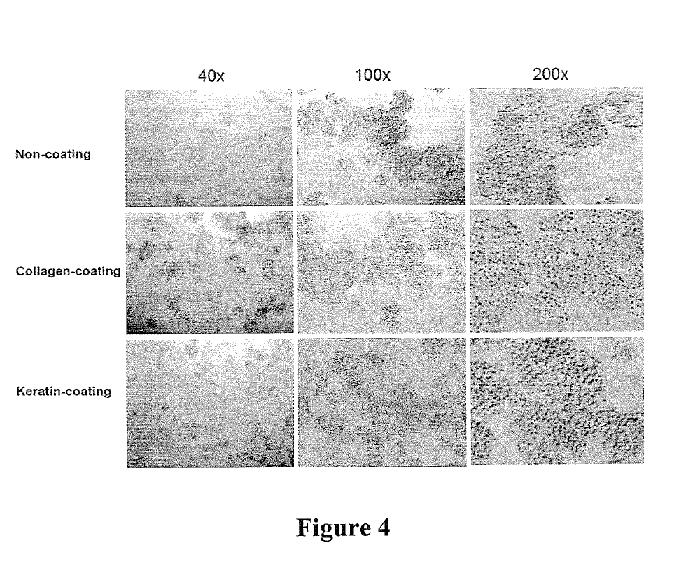

A 100 μg/ml keratin solution was prepared by diluting 0.5 ml of a 10 mg/ml keratin gel stock solution 100× by addition of 50 ml of deionized water to make 50 ml of a 100 μg/ml working solution. The working solution was filtered through a 0.4 μm filter, and then filtered through a 0.22 μm filter. The solution was then used to coat the wells of a 96-well plate (30 μl solution) or a 24-well plate (200 μl solution). Plates were incubated for 48 hours at 37° C., during which time the keratin adhered to the well. Excess coating solution was removed, and the wells were washed with phosphate buffered saline (PBS) twice. Plates were covered to keep the wells from drying and stored at 4° C. Wells were washed with PBS before seeding with cells. An acidic collagen coating was similarly prepared. Beta TC-6 cells (insulin-producing cells from mouse with insulinoma) were used to measure cell adhesion and proliferation (FIGS. 3-4).

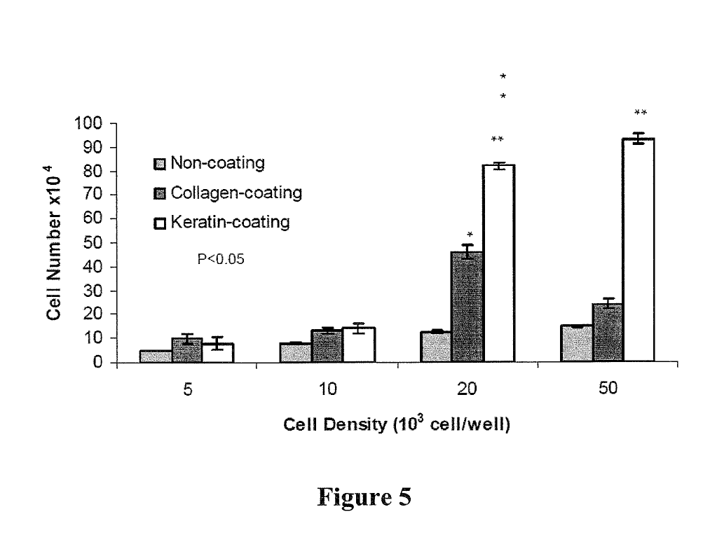

Results: Beta TC-6 cells were able to attain a higher density with the keratin-coated plates than the collagen-coated and non-coated plates (FIG. 5). Different solutions—PBS, acetic acid and distilled water—were used to make the dilution from the stock into the working solution for coating. Observations are summarized in Table 1.

TABLE 1
Beta TC-6 Cell Growth at Day 7 in Different Solutions.

| | Solution | | | | | | | | |
|---|---|---|---|---|---|---|---|---|---|
| | | | | | | | Acetic | Acetic | |
| | PBS | PBS | | ddw | ddw | | acid | acid | |
| Coating | Keratin | Collagen | Non | Keratin | Collagen | Non | Keratin | Collagen | Non |
| materials | | | | | | | | | |
| Dish | Brown | Brown | Clear | Brown | Brown | Clear | Clear | Clear | Clear |
| background | fragment | fragment | | fragment | Fragment | | | | |
| Cell growth | small | large | small | large | large | small | large | large | small |
| pattern | | | | | | | | | |
| Cell number | 15.03 | 10.67 | 12.95 | 12.63 | 12.25 | 12.76 | 14.62 | 13.05 | 11.57 |
| ×10⁴/ml | | | | | | | | | |

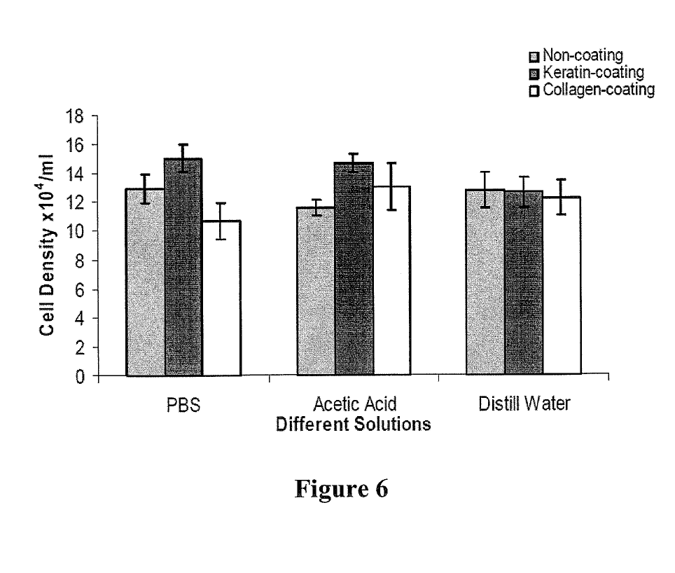

This variation in solution had no significant effect on cell density (FIG. 6). (Note, however, that in some instances acetic acid should not be used because it may fractionate the material during coating.)

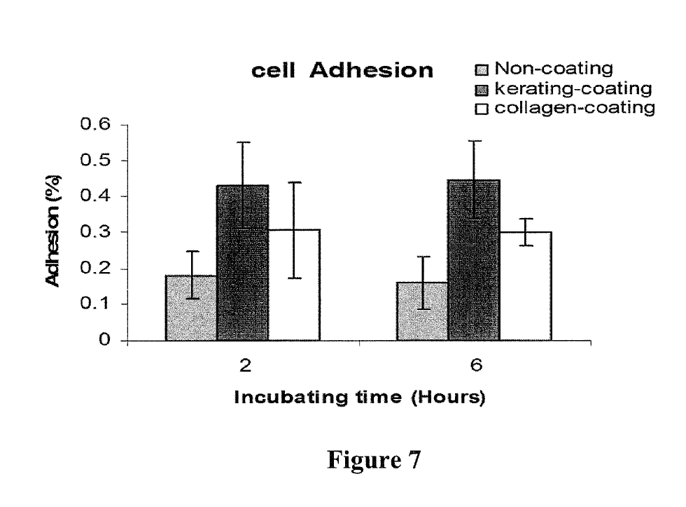

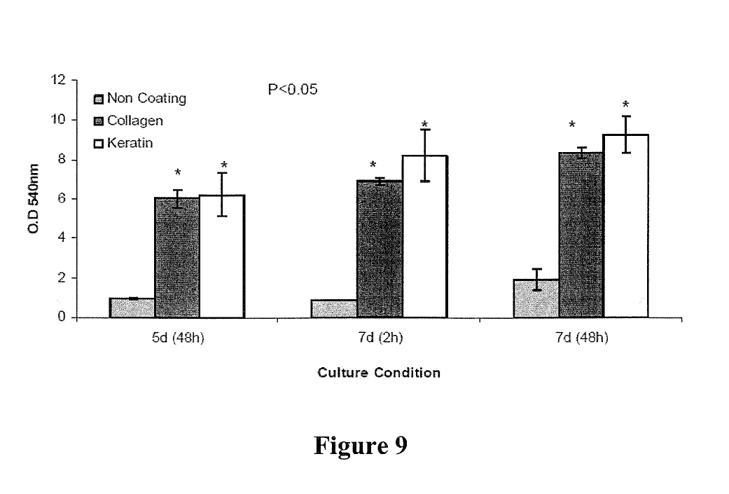

Cell adhesion in the keratin-coated plates was significantly higher than the non-coated plates, but not significantly different from the collagen-coated plates (FIG. 7). Insulin secretion by the Beta TC-6 cells grown on both keratin-coated and collagen-coated plates was significantly higher than non-coated plates (FIG. 9).

### EXAMPLE 4

### Cell Culture with Keratin Microparticles

1. Remove media from the source culture flask or dish.

2. Rinse with 5 mL of Dulbecco's Phosphate-Buffered Saline (D-PBS) and remove.

3. Add 1 mL of pre-warmed 0.05% Trypsin-EDTA to the flask.

4. Incubate until the cells have detached (about 5 to 10 minutes at room temperature).

5. Add 5 mL of growth medium containing 500 μg/mL Soybean Trypsin Inhibitor to the flask and gently triturate. Transfer the cell suspension to a 15 mL centrifuge tube.

6. Determine viable and total cell counts.

7. Centrifuge the cells for 10 minutes at 100×g.

8. Aspirate the supernatant and gently resuspend the cell pellet in the desired volume of pre-warmed, complete growth medium.

9. Transfer the cells to a spinner flask containing the desired amount of microparticles (cells should be seeded at a sub-confluent density so as not to induce contact inhibition, if desired). Put the flask in an incubator with caps loosened to allow for oxygenation/aeration and gently agitate.

10. Alternately, microparticles can be spread on the bottom of a culture flask and cells seeded statically on top or some combination of 9 and 10.

11. Replace media with fresh, complete growth media every 2 to 3 days by first allowing the microparticles to settle and then aspirating and replacing the majority of the supernatant.

The foregoing is illustrative of the present invention, and is not to be construed as limiting thereof. The invention is defined by the following claims, with equivalents of the claims to be included therein.

That which is claimed is:

1. A cell culture microcarrier comprising a keratin in porous particulate form.

2. The microcarrier of claim 1, wherein said microcarrier has an average diameter greater than 10 micrometers.

3. The microcarrier of claim 1, wherein said microcarrier has an average diameter between 10 micrometers and 500 micrometers.

4. The microcarrier of claim 1, wherein said keratin is selected from the group consisting of: alpha kerateines, gamma kerateines, and combinations thereof.

5. The microcarrier of claim 1, wherein said keratin comprises a meta keratin.

6. The microcarrier of claim 1, wherein said keratin consists essentially of a meta keratin.

7. The microcarrier of claim 1, wherein said keratin is acidic or basic.

8. The microcarrier of claims 1, further comprising a liquid carrier.

9. The microcarrier of claim 1, further comprising viable cells attached thereto.

10. A cell culture substrate comprising a keratin coating.

11. The substrate of claim 10, wherein said keratin is selected from the group consisting of: alpha kerateines, gamma kerateines, and combinations thereof.

12. The substrate of claim 10, wherein said keratin comprises a meta keratin.

13. The substrate of claim 10, wherein said keratin consists essentially of a meta keratin.

14. The substrate of claim 10, wherein said keratin is acidic or basic.

15. The substrate of claim 10 further comprising a liquid carrier.

16. The substrate of claim 10 further comprising viable cells attached thereto.

17. A method of administering cultured cells, comprising administering the microcarrier of claim 1 to a subject in need thereof.

18. The method of claim 17, wherein said administering step is carried out by injection.

19. A kit comprising:
(a) a suitable container;
(b) a plurality of cell culture microcarriers according to claim 9 packaged into said container; and
(c) optionally, instructions for use.

20. The kit of claim 19, wherein said microcarriers are packaged in said container in sterile form.

21. The kit of claim 19, wherein said microcarriers are provided in a liquid carrier.

22. The kit of 21, wherein said liquid carrier comprises an alcohol.

23. The kit of claim 19, wherein said container comprises an ampule.
